(19) United States
(12) Patent Application Publication
(10) Pub. No.: US 2009/0125086 A1
(43) Pub. Date: May 14, 2009

(54) Thermotherapy Device

(75) Inventors: Tetsuro Juta, Osaka (JP); Rikako Akita, Osaka (JP); Naoki Dobashi, Osaka (JP); Goro Sonada, Hyogo (JP)

Correspondence Address: KRATZ, QUINTOS & HANSON, LLP, 1420 K Street, N.W., Suite 400, WASHINGTON, DC, 20005, US

(73) Assignee: KOBAYASHI PHARMACEUTICAL CO., LTD.,, OSAKA-SHI (JP)

(21) Appl. No.: 11/988,140

(22) PCT Filed: Sep. 15, 2005
(86) PCT No.: PCT/JP2005/017039

§ 371 Date: Dec. 31, 2007

(30) Foreign Application Priority Data
Jul. 4, 2005 (JP) 2005-005204

(51) Int. Cl. A61F 7/08

(52) U.S. Cl. 607/108; 607/114

(57) ABSTRACT

A thermotherapy device worn on a joint that includes a tubular holder composed of a stretchable material and a chemical warmer held in the holder. The holder has an accommodating portion in which the chemical warmer can be placed and removed therefrom, or to which the chemical warmer can be attached. The thermotherapy device can maintain temperatures suitable for thermotherapy for a long period, remains in place despite the motion of the joint, and can hold a heat-generating member in a suitable area. The thermotherapy device is thus effective for use on joints.

### TECHNICAL FIELD

The present invention relates to a thermotherapy device utilizing chemical warmers for use in thermally treating joints.

### BACKGROUND ART

Chemical warmers that utilize the heat of oxidation generated by metals such as iron, which are so-called disposable warmers, have been developed for the chief purpose of easily providing warmth. Various types of chemical warmers are commercially available or proposed to suit the area to which the chemical warmer is applied, such as a type held in the hand, a type put in pockets of clothing, a type attached to clothing or the skin, a type laid on shoe soles, a type attached to the sole of the feet (socks), a belt-shaped type that is wound around the area to be warmed, and the like.

Known chemical warmers have thus been made on the premise that they are used in a stationary state. Therefore, known forms of chemical warmers are not suitable for warming movable parts (joints), such as the knee, elbow, wrist, and ankle. They are especially difficult to fix onto the knee and elbow, which are capable of a wide range of motion.

Chemical warmers that are wound like a belt have recently been proposed (Japanese Unexamined Patent Application Publication No. 2002-146612). These chemical warmers, however, use fixing tools that are warming tools themselves, and cover a wide range of regions including those other than a particular afflicted area, thus resulting in overheating. They can be used, therefore, only in limited seasons and places. Moreover, further improvements are demanded in these chemical warmers in that although they are effective to some degree against rotational movement of the wrist, ankle, or neck, they cannot conform to considerable bending or to the expansion and contraction of the elbow or knee, and deviate in position.

Furthermore, as described above, because known chemical warmers are intended for providing warmth, heating at a relatively high temperature for a long period has been sought with a view toward preventing low-temperature burns.

Therefore, when known chemical warmers are applied to thermotherapy, which principally involves treatments at relatively low temperatures, the heating temperature may exceed the temperature necessary for the intended treatment, thus raising concern for low-temperature burns caused by the careless application of such a known chemical warmer to thermotherapy. While some proposals have previously been made (e.g., Japanese Unexamined Utility Model Application Publication No. 1997-62820), further research is needed before such proposals are put into practical use.

The object of the present invention is to provide a thermotherapy device for a joint that can maintain temperatures suitable for thermotherapy for a long period, remain in place despite the motion of the joint, and can hold a heat-generating member in a suitable area.

### DISCLOSURE OF THE INVENTION

The present invention relates to a thermotherapy device worn on a joint that includes a tubular holder composed of a stretchable material and a chemical warmer held in the holder; the holder having an accommodating portion in which the chemical warmer can be placed and removed therefrom, or to which the chemical warmer can be attached.

The thermotherapy device worn on a joint according to the invention encompasses a thermotherapy device for the knee or elbow, which are capable of a wide range of motion (a first aspect of the invention), and a thermotherapy device for the wrist or ankle (a second aspect of the invention). These inventions will be individually described below.

The first aspect of the present invention relates to a thermotherapy device worn on the knee or elbow that includes a tubular holder composed of a stretchable material and a chemical warmer held in the holder; the holder having an accommodating portion in which the chemical warmer can be placed and removed therefrom, or to which the chemical warmer can be attached; and the thermotherapy device being worn on the knee or elbow so that the chemical warmer accommodating portion is positioned around the leg or arm.

The pressure applied to the skin when the holder is worn on the knee or elbow with the chemical warmer being accommodated in the holder is preferably 80 hPa or less, so as to prevent the holder from applying excessive pressure to the leg or arm.

The holder may be composed of a sheet of a stretchable textile product. When the holder has a bag shape with a multilayer structure, the chemical warmer accommodating portion may be an integral part of the bag.

The chemical warmer accommodating portion may be formed without any partition provided in the bag-shaped holder, or may be a pocket formed with a partition in the bag-shaped holder. When the chemical warmer accommodating portion is formed without any partition provided in the bag-shaped holder, the position of the chemical warmer can be freely changed or adjusted for convenience.

The chemical warmer, which serves as a heat-generating member, may have an inner bag in which only one surface is air-permeable, or may have an inner bag in which both surfaces are air-permeable.

The second aspect of the present invention relates to a thermotherapy device worn on the wrist that includes a tubular holder composed of a stretchable material and a chemical warmer held in the holder; the holder having a wrist region and a palm-and-back-of-the-hand region; the wrist region, for example, of the holder having an accommodating portion in which the chemical warmer can be placed and removed therefrom, or to which the chemical warmer can be attached; and the palm-and-back-of-the-hand region having an opening through which a finger is inserted.

The second aspect of the present invention also relates to a thermotherapy device worn on the ankle that includes a tubular holder composed of a stretchable material and a chemical warmer held in the holder; the holder having an ankle region and a back-of-the-foot-and-heel region; and the ankle region, for example, of the holder having an accommodating portion in which the chemical warmer can be placed and removed therefrom, or to which the chemical warmer can be attached.

The pressure applied to the skin when the holder is worn on the wrist or ankle with the chemical warmer being accommodated in the holder is preferably 80 hPa or less, so as to prevent the holder from applying excessive pressure to the wrist or ankle.

The holder may be composed of a sheet of a stretchable textile product. When the holder has a bag shape with a multilayer structure, the chemical warmer accommodating portion may be an integral part of the bag.

The chemical warmer accommodating portion may be formed without any partition provided in the bag-shaped holder, or may be a pocket formed with a partition in the bag-shaped holder. When the chemical warmer accommodating portion is formed without any partition provided in the bag-shaped holder, the position of the chemical warmer can be freely changed or adjusted for convenience.

The chemical warmer, which serves as a heat-generating member, may have an inner bag in which only one surface is air-permeable, or may have an inner bag in which both surfaces are air-permeable.

### BRIEF DESCRIPTION OF THE DRAWINGS

FIG. 1 shows a schematic plan view of a preferred embodiment of the thermotherapy device for the elbow of the invention;

FIG. 2 shows a cross-sectional view taken along the line C-C of FIG. 1;

FIG. 3 shows a cross-sectional view taken along the line D-D of FIG. 1;

FIG. 4 shows a schematic plan view of a portion near an opening of the holder shown in FIG. 1;

FIG. 5 shows a schematic diagram for use in illustrating the thermotherapy device of FIG. 1 worn on the elbow;

FIG. 6 shows a schematic plan view of another preferred embodiment of tightening portions in the thermotherapy device for the elbow of the invention;

FIG. 7 shows a schematic plan view of still another preferred embodiment of tightening portions in the thermotherapy device for the elbow of the invention;

FIG. 8 shows a schematic plan view of yet another embodiment of tightening portions of the thermotherapy device in the thermotherapy device for the elbow of the invention;

FIG. 9 shows a partially schematic side view for use in illustrating a closed state of a mechanism for adjusting the width of the thermotherapy device for the elbow of the invention;

FIG. 10 shows a partially schematic side view for use in illustrating an open state of the mechanism for adjusting the width of the thermotherapy device for the elbow of the invention;

FIG. 11 shows a schematic plan view for use in illustrating a mechanism for adjusting the longitudinal size of the thermotherapy device for the elbow of the invention;

FIG. 12 shows a schematic plan view of another preferred embodiment of the thermotherapy device for the elbow of the invention;

FIG. 13 shows a schematic cross-sectional view of an example of a chemical warmer that can be used in the invention;

FIG. 14 is a graph depicting changes in the skin temperature measured in Example 1;

FIG. 15 is a schematic perspective view of a preferred embodiment of the thermotherapy device for the wrist of the invention;

FIG. 16 shows a schematic diagram for use in illustrating the thermotherapy device for the wrist shown in FIG. 15 worn on the wrist;

FIG. 17 shows a schematic perspective view of another preferred embodiment of the thermotherapy device for the wrist of the invention;

FIG. 18 shows a cross-sectional view taken along the line A-A of FIG. 16;

FIG. 19 shows a schematic cross-sectional view of an example of a chemical warmer that can be used in the invention;

FIG. 20 shows a schematic plan view of a preferred embodiment of the thermotherapy device for the ankle of the invention;

FIG. 21 shows a schematic diagram for use in illustrating the thermotherapy device for the ankle shown in FIG. 20 worn on the ankle; and

FIG. 22 is a graph depicting changes in the skin temperature measured in each of Examples 2 and 3.

### BEST MODE FOR CARRYING OUT THE INVENTION

The first aspect of the present invention relates to a thermotherapy device to be worn on the knee or elbow that comprises a tubular holder composed of a stretchable material and a chemical warmer held in the holder; the holder having an accommodating portion in which the chemical warmer can be placed and removed therefrom, or to which the chemical warmer can be attached; and the thermotherapy device being worn on the knee or elbow so that the chemical warmer accommodating portion is positioned around the leg or arm.

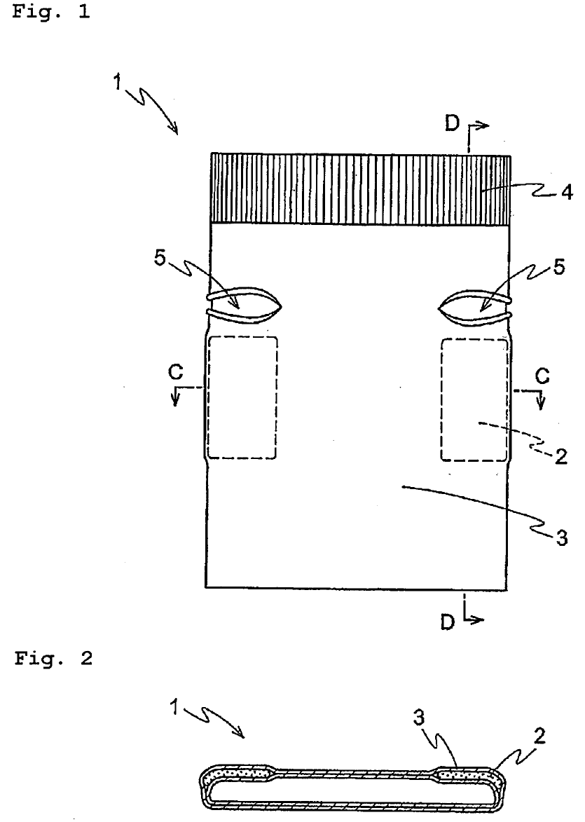

Referring to FIGS. 1 to 5, a preferred embodiment of the thermotherapy device for the elbow according to the invention will be described. The present invention, however, is not limited to the embodiment, and various modifications can be made thereto. Needless to say, the embodiment can also be implemented as a thermotherapy device for the knee by making modifications to the dimensions and the like.

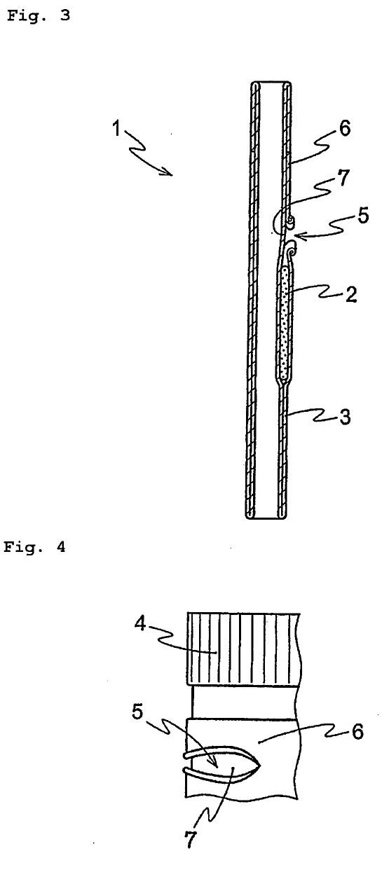

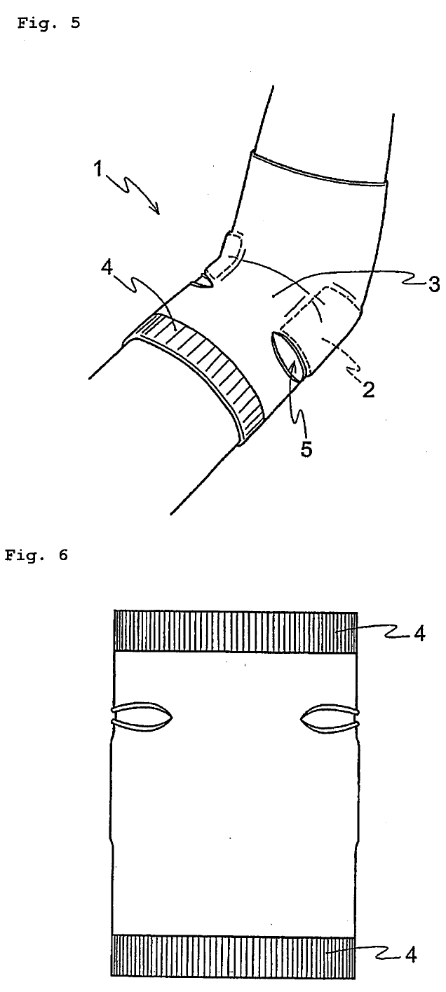

FIG. 1 shows a schematic plan view of the thermotherapy device for the elbow of the invention; FIG. 2 shows a cross-sectional view taken along the line C-C of FIG. 1; FIG. 3 shows a cross-sectional view taken along the line D-D of FIG. 1; FIG. 4 shows a schematic plan view of a portion near the tightening portion and an opening; and FIG. 5 shows a schematic diagram for use in illustrating the thermotherapy device of FIG. 1 worn on the elbow.

The thermotherapy device comprises a tubular holder 1 and chemical warmers 2. The holder 1 comprises a bag-shaped accommodating portion 3 in which the chemical warmers 2 can be placed and removed therefrom. In this embodiment, the chemical warmer accommodating portion 3 has two openings through which each chemical warmer 2 can be placed and removed, and the chemical warmers 2 accommodated in the accommodating portion 3 can be freely positioned therein.

The holder 1 is composed of a stretchable material so as to conform to the elbow joint when it is bent and stretched. When necessary, a tightening portion 4 may also be provided in a portion of the holder 1 to facilitate fixing of the holder 1 (see FIG. 1).

As mentioned above, if the holder 1 fits too tightly around the arm, the blood flow may stop and cause the congestion of blood. Therefore, the pressure applied to the skin when the holder 1 is worn on the knee or elbow with the chemical warmers 2 being accommodated in the accommodating portion 3 is preferably set to 80 hPa or less. If the congestion of blood occurs, heat will be accumulated in the afflicted area, possibly resulting in a low-temperature burn; therefore, the pressure is more preferably 40 hPa or less, and still more preferably 35 hPa or less. If the holder 1 becomes loose, it is difficult to position the holder 1 on the area to be treated because the holder 1 loses conformity to the afflicted area and easily moves; therefore, the lower limit of the pressure is 10 hPa, preferably 20 hPa, and more preferably 30 hPa.

In this embodiment, the holder 1 has a bag shape with a multilayer structure, in which a sheet of a stretchable textile product (such as a woven, knitted, nonwoven, or like cloth) is folded into two layers, and the open regions are closed. The openings 5 are provided in the knitting process. While the number of openings 5 may be two as in this embodiment, it may be one to six, and preferably one to three, depending on the area to be treated or the position thereof. While the positions of openings 5 are not particularly limited, when the holder 1 is great in length or width, or each chemical warmer 2 is small, they may be arranged vertically or horizontally in the longitudinal direction and/or lateral direction of the accommodating portion 3, or the positions may be diagonally staggered.

As shown in FIG. 4, the opening 5 is made in the knitting process by leaving open a portion of an outer cloth 6 of the bag-shaped holder 1. In this case, the accommodating portion 3 is a space between the outer cloth 6 and an inner cloth 7 that constitute the holder 1, in which the chemical warmers 2 are accommodated.

Although not illustrated, the bag-shaped holder 1 may be sewn appropriately so that an individual accommodating portion (pocket) is formed. In this case, if the pocket is disposed on an inner or outer bending portion of the joint, the wearer will feel pressure during joint flexure, so that the pocket is preferably formed on a side portion of the arm (or the leg for use on the knee) so as not to prevent the motion of the joint. Depending on the area to be treated, however, a pocket may additionally be disposed so as to surround the arm, and/or may be disposed in the longitudinal direction of the arm.

Moreover, although not illustrated, an accommodating portion may be prepared separately, and then sewn on or attached to the holder 1. When attaching an accommodating portion, an adhesive may be used, or hook-and-loop fasteners or the like may be used so as to allow the position of the accommodating portion to be detachably changed.

In addition, each opening 5 may be provided with a lid or a cover, or may be provided with buttons or fasteners (zippers or hook-and-loop fasteners) in order to prevent the chemical warmer from dropping off.

The holder 1 is composed of a stretchable material. The material used may be composed of a synthetic fiber alone or a mixture of synthetic fibers and natural fibers, such as vegetable fibers, animal fibers, mineral fibers, and the like. Such materials may be suitably selected in consideration of stretchability, wearing comfort, heat conductivity, workability, wear resistance, chemical resistance, and the like. Examples of preferable fiber materials include polyamide, polyurethane, polyester and the like. Although not limited, the thickness of the fiber may be from 3 to 100 μm, and preferably from 10 to 50 μm. Mechanical properties of the fiber are preferably such that the tensile strength is from 10 to 300 kg/mm², and preferably from 10 to 100 kg/mm², and the elongation percentage is from 1 to 50%, and preferably from 5 to 30%.

While the stretchability also depends on the properties of the fiber, it can be controlled by selecting the method of cloth production. The cloth is preferably knitted by a method such as circular knitting, weft knitting, plain knitting, rib knitting, purl knitting, interlock knitting or the like, with circular knitting being particularly preferable because a circular-knitted cloth can be preformed into a tubular shape without sewing.

The holder 1 is prepared using the above-described stretchable material. Any method can be used for preparing the holder 1 other than the above-described method of using a single sheet or using a bag-shaped sheet, as long as stretchability and flexibility are not impaired.

Moreover, in order to improve the wearing comfort and softness to the skin, a backlining layer (not illustrated) composed of a cotton (towel) fabric, a linen fabric, or a silk fabric may be formed on the body-side of the inner cloth 7 of the holder.

The tightening portion 4 is preferably prepared by using the same fabric as that of the holder, but changing the knitting method to impart a tightening force, or by using a rubber material. The method is not particularly limited, however, as long as it can impart a tightening force. For example, hook-and-loop fasteners or a tightening belt, or a combination of these may alternatively be used in place of the above-mentioned methods.

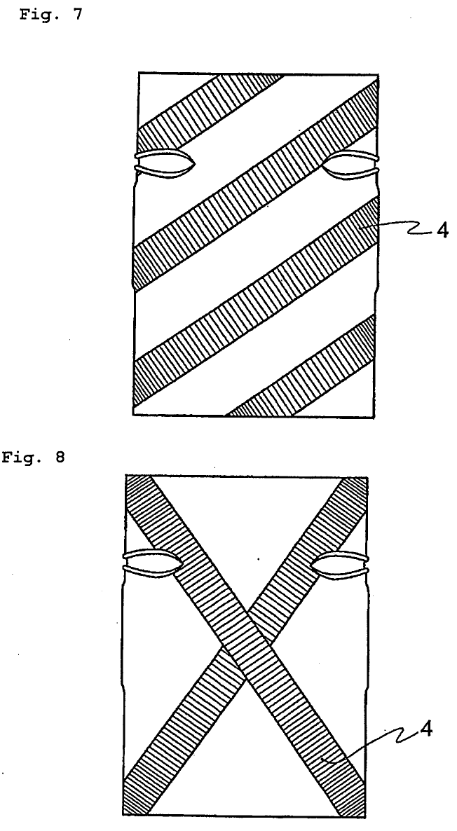

The tightening portion 4 may be formed on one end of the holder 1, as shown in FIG. 1. Alternatively, as shown in FIG. 6, tightening portions 4 may be formed on both ends of the holder 1. As another alternative, as shown in FIG. 7, tightening portions 4 may be spirally formed around the holder 1. As yet another alternative, as shown in FIG. 8, tightening portions 4 may be formed diagonally in a crisscross fashion.

The holder 1 may be made available in various sizes so that a suitable size can be selected according to the body size of a potential user.

Alternatively, as shown in FIGS. 9 to 11, the holder 1 may comprise a mechanism for adjusting the size of the holder according to the body size of the user.

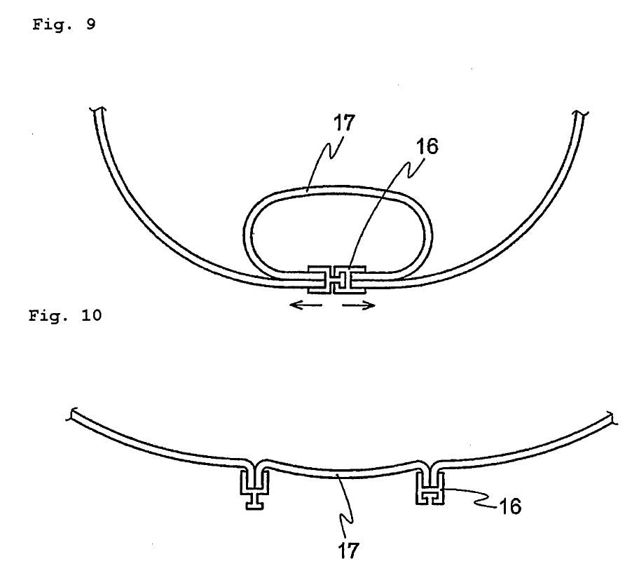

FIGS. 9 and 10 show schematic views of one embodiment of the invention in which a mechanism for adjusting the size of the holder in the width (diameter) direction is provided at a position in the longitudinal direction of the holder. The mechanism includes an extension cloth 17, and the diameter of the holder can be increased by opening the fastener 16.

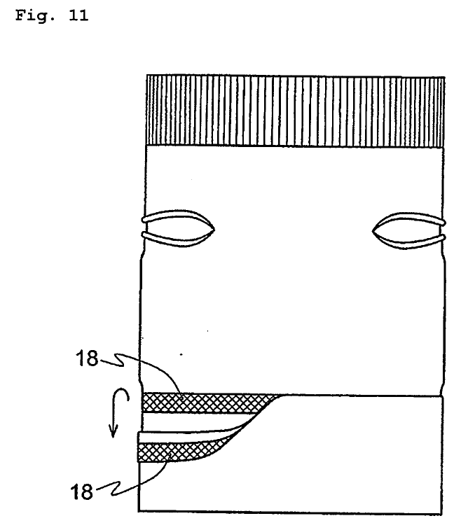

FIG. 11 shows a schematic plan view of one embodiment of the invention in which the longitudinal length of the holder can be adjusted. In this embodiment, the length can be adjusted by fastening hook-and-loop fasteners 18 of the holder.

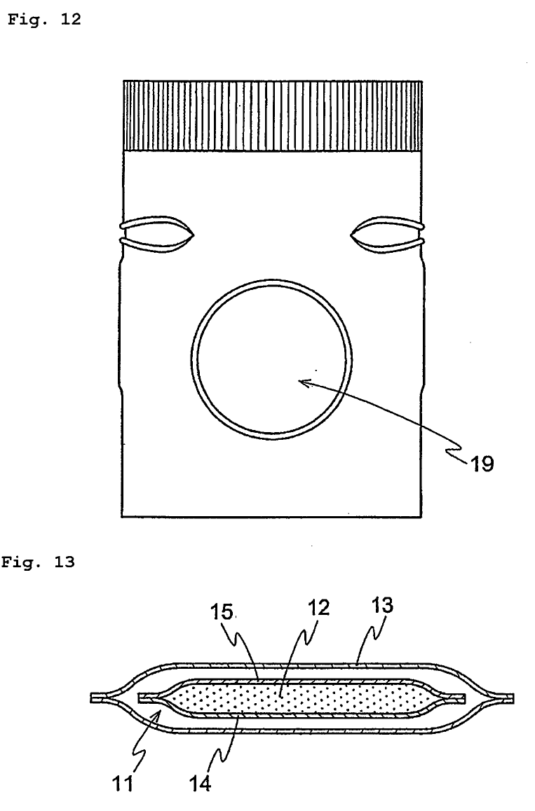

Moreover, as shown in FIG. 12, because the joint protrudes when the elbow or knee is bent, a hole 19 may be preformed in a region of the holder corresponding to the protrusion, so as to facilitate bending of the elbow or knee.

The chemical warmer used as another element of the invention is now described referring to FIG. 13.

For example, as shown in FIG. 13, the chemical warmer 2 used in the invention comprises a flat bag (hereinafter referred to as an “inner bag”) 11; an exothermic composition 12 that is sealed in the inner bag 11 and that oxidizes and generates heat in the presence of air; and a hermetic bag (hereinafter referred to as an “outer bag”) 13 housing the flat bag 11.

The exothermic composition 12 is preferably dispersed in the inner bag 11 in an amount of 0.1 to 1 g per 10 mm square so as to achieve even and mild heating.

The outer bag 13 is preferably composed of a porous film such as polyethylene, polypropylene, a silica-evaporated film, a vinylidene chloride coating film or the like. Among these examples, a vinylidene chloride coating film is preferred in view of its impermeability to oxygen and water vapor and an appropriate permeability to hydrogen. The thickness of the porous film is preferably from 30 to 300 μm.

A known exothermic composition may be used as the exothermic composition 12 that is sealed inside the flat bag 11 and that oxidizes and generates heat in the presence of air. Examples of usable exothermic compositions include, but are not limited to, those comprising metallic particles such as iron particles, activated carbon, water, water-holding agents (such as wood flour, vermiculite, diatomite, perlite, silica gel, alumina, water-absorbing resins and the like), salt, and the like.

At least one portion of the inner bag 11 is an air-permeable surface with a group of small air holes or micropores. That is to say, one surface of the inner bag 11 (the lower surface in FIG. 13) may be an air-permeable surface 14, and the other surface may be an air-impermeable surface 15, or both the surfaces may be air-permeable. Alternatively, a portion of one surface of the inner bag 11 may be an air-permeable surface, with the remaining portion being an air-impermeable surface.

When an air-impermeable surface is provided, it is preferably composed of, for example, a synthetic resin, such as nylon, polyethylene, polypropylene or the like.

The air-permeable surface is composed of a known material, such as a woven cloth, a nonwoven cloth, a knitted cloth, a perforated film of any of the aforementioned air-impermeable materials, or a combination thereof.

In the invention, the size of the inner bag 11 is not particularly limited, and may be selected to suit the shape, size or the like of the chemical warmer accommodating portion (or pocket). The shape of the inner bag 11 is also not limited to a rectangular shape, but may be circular, oval, flap-shaped, heart-shaped, or the like. The thickness of the inner bag 11 is from 1 to 15 mm, and preferably 2 to 8 mm, so as to provide satisfactory comfort while not likely to hinder the motion of the joint. The thickness of the inner bag 11 may be even, or may be uneven to vary the feeling of pressure.

The air permeability of the inner bag 11 may be determined in consideration of the skin surface temperatures at which so-called low-temperature burns may occur and the time therefor. For example, low-temperature burns may occur in six hours at a skin temperature of 42° C., in three hours at a skin temperature of 43° C., in 1.5 hours at a skin temperature of 44° C., and in 45 minutes at a skin temperature of 45° C. The air permeability may be controlled while considering the composition of the exothermic composition so that these thresholds are not exceeded. In the case of short-period treatments, however, the heating temperature may further be elevated (for example, to 60° C., or even higher to 70° C.).

While the air permeability of the entire inner bag 11 is not limited, the water vapor permeability as measured according to Method A (Humidity Sensor Method) defined in JIS (Japanese Industrial Standard) K 7129 is preferably 100 to 2,000 g/m²·24 h, and more preferably 200 to 800 g/m²·24 h. If the water vapor permeability exceeds 2,000 g/m²·24 h, the exothermic composition may generate excessive heat, whereas if it is lower than 100 g/m²·24 h, the exothermic composition may not be able to generate sufficient heat.

The heating temperature and duration can be controlled by changing the structures of the chemical warmer 2 and the accommodating portion 3, and in particular, the structures of the inner (the arm- or knee-side) cloth 7 and the outer cloth 6 of the accommodating portion 3, and also by changing the material of the backlining layer.

Examples of structural combinations include, but are not limited to, the following.

(1) Cases Where the Inner Cloth Is Air-Permeable, and the Chemical Warmer Is Air-Permeable on Both Surfaces Thereof In these cases, a relatively high heating temperature is achieved, but care needs to be taken concerning low-temperature burns and the like. When necessary, a backlining layer may be provided to further control the air permeability.

(2) Cases Where the Inner Cloth Is Air-Permeable, and the Chemical Warmer Is Air-Permeable on Only One Surface Thereof In these cases, air intake is facilitated by placing the chemical warmer in the accommodating portion 3 so that the air-permeable surface is positioned on the side of the accommodating portion opposite to the body (outer side), thereby making the heating temperature relatively high (about 38 to about 42° C.). In contrast, air intake is restricted by placing the chemical warmer in the accommodating portion 3 so that the air-permeable surface is positioned on the body side (inner side) of the accommodating portion, thereby making the heating temperature relatively low (about 36 to about 40° C.).

That is to say, the heating temperature can be adjusted to a temperature necessary for thermotherapy by changing the direction in which the chemical warmer 2 is placed.

(3) Cases Where the Inner Cloth Is Air-Impermeable or Substantially Air-Impermeable, and the Chemical Warmer Is Air-Permeable on Both Surfaces Thereof In these cases, a relatively high heating temperature can be achieved, and the heating temperature can be controlled by selecting the heat conductivity, thereby avoiding low-temperature burns and the like.

(4) Cases Where the Inner Cloth Is Air-Impermeable or Substantially Air-Impermeable, and the Chemical Warmer Is Air-Permeable on Only One Surface

In these cases, as in cases (2) above, the heating temperature can be adjusted to a temperature necessary for thermotherapy by changing the direction in which the chemical warmer 2 is placed, while considering the heat conductivity of the inner cloth.

(5) Cases Where Both the Inner Cloth and the Outer Cloth Are Air-Impermeable, and Means for Placing and Removing the Chemical Warmer Is Provided to the Accommodating Portion In these cases, the function of the accommodating portion enables temperature adjustments necessary for thermotherapy.

Examples of means to make the inner cloth air-impermeable or substantially air-impermeable include attaching an air-impermeable film to the body side of the accommodating portion.

In one embodiment, the chemical warmer may be simply attached (fastened) to the holder, rather than making the accommodating portion of the chemical warmer into a bag shape (or pocket) as described above. In this case, the chemical warmer is attached to the outer or inner surface of the holder. Examples of means for attaching the chemical warmer include attaching with adhesive, fixing with hook-and-loop fasteners, fixing with buttons, and the like.

When a chemical warmer is attached to the holder, an adhesive layer composed of a common adhesive material may be formed on one surface of the inner bag 11 (for example, on the surface 14 in FIG. 13), and a release paper or a release film may be formed on the adhesive layer.

The adhesive layer may be formed entirely on one surface of the inner bag 11, or may be partially formed in suitable patterns such as stripes, checks, dots or the like. The adhesive layer may also be formed on either an air-impermeable surface or an air-permeable surface.

Although a thermotherapy device for use on the elbow has been described above, it can also be used for the knee by adjusting the size to the knee.

The second aspect of the present invention will next be described in detail.

A thermotherapy device for the wrist encompassed in the second aspect of the present invention comprises a tubular holder composed of a stretchable material and a chemical warmer held in the holder; the holder comprising a wrist region and a palm-and-back-of-the-hand region; the wrist region having an accommodating portion in which the chemical warmer can be placed and removed therefrom, or to which the chemical warmer can be attached; and the palm-and-back-of-the-hand region having an opening through which a finger is inserted.

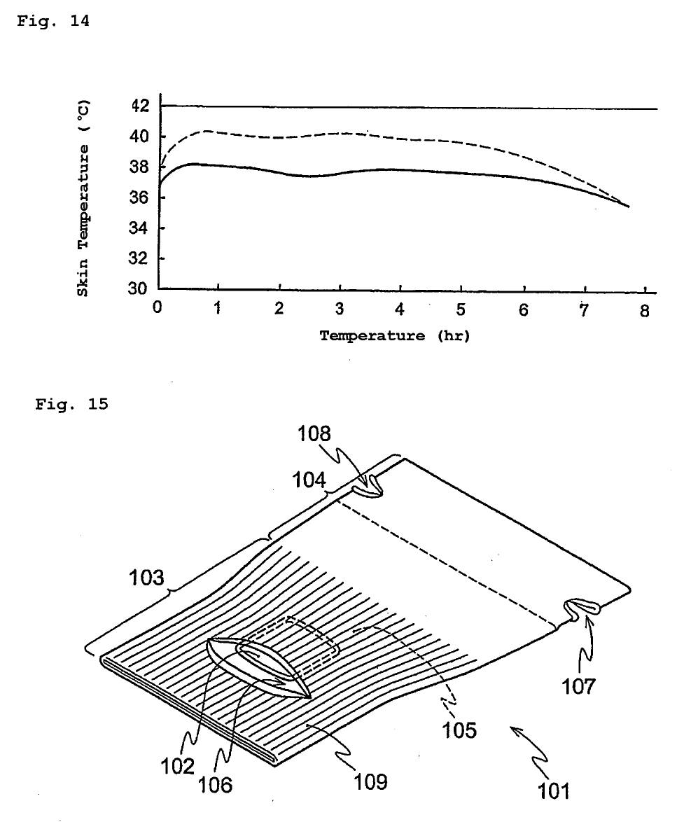

Referring to FIGS. 15 to 18, a preferred embodiment of the thermotherapy device for the wrist according to the invention will be described. The present invention, however, is not limited to this embodiment, and various modifications can be made thereto. Needless to say, the embodiment can also be implemented as a thermotherapy device for the ankle by adding modifications as described below.

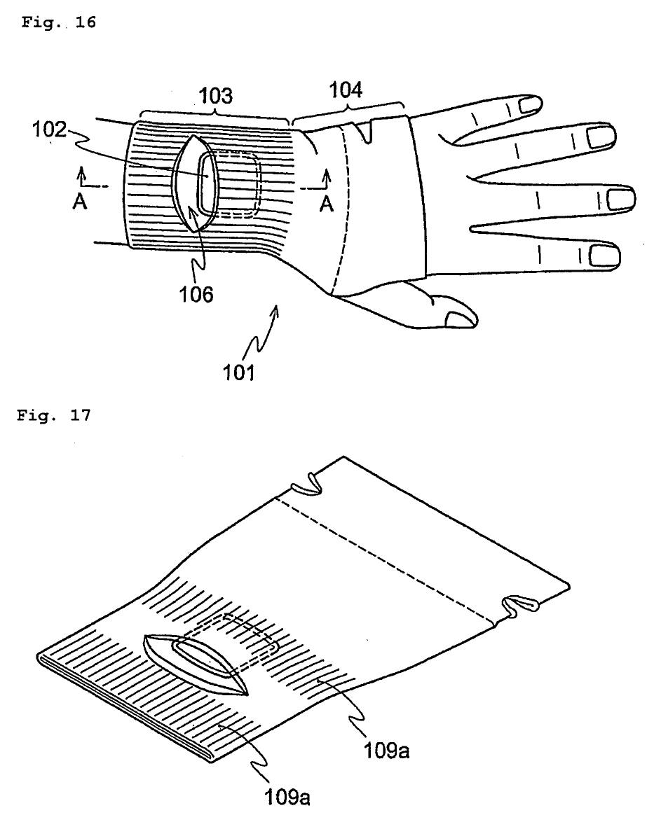

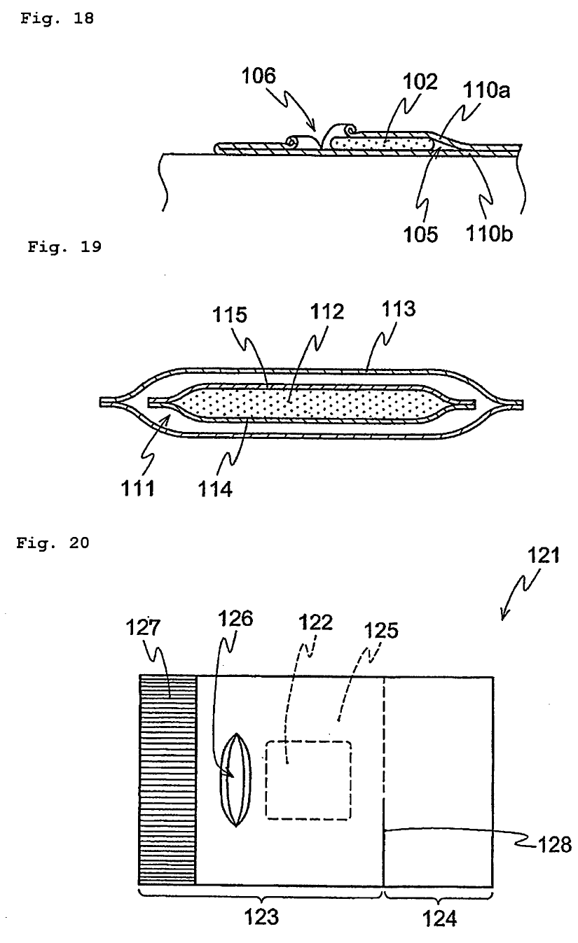

FIG. 15 shows a schematic perspective view of the thermotherapy device for the wrist of the invention; FIG. 16 shows a schematic diagram for use in illustrating the thermotherapy device of FIG. 15 worn on the wrist; FIG. 17 shows a schematic perspective view showing another embodiment of the thermotherapy device for the wrist of the invention; and FIG. 18 shows a cross-sectional view taken along the line A-A of FIG. 16.

The thermotherapy device comprises a tubular holder 101 and a chemical warmer 102. The holder 101 has a wrist region 103 and a palm-and-back-of-the-hand region (hereinafter referred to as a “back region”) 104.

The wrist region 103 of the holder 101 has a bag-shaped accommodating portion 105 in which the chemical warmer 102 can be placed and removed therefrom. In this embodiment, the chemical warmer accommodating portion 105 has one opening 106 through which the chemical warmer 102 can be placed and removed, and the chemical warmer 102 accommodated in the accommodating portion 105 can be freely positioned therein.

The back region 104 of the holder 101 has insertion openings 107, 108 through which fingers are inserted. The insertion opening 107 is an opening for inserting the left thumb for thermotherapy of the left wrist (see FIG. 16), and the insertion opening 108 is an opening for inserting the right thumb for application to the right hand. The number of inserted fingers is not particularly limited, and hence, the number and the positions of insertion openings 107, 108 are also not limited. Moreover, the thermotherapy device for the wrist according to the second aspect of the invention may be provided as an extension portion of a glove.

The holder 101 is composed of a stretchable material so as to conform to the wrist joint when it is bent and stretched. In the second aspect of the invention, a tightening portion 109 is provided in the wrist region of the holder 101, so as to facilitate fixing of the holder 101 (see FIG. 15). The accommodating portion 105 for the chemical warmer 102 is preferably formed in the tightening portion 109. As shown in FIG. 17, however, tightening portions may also be formed as annular regions 109a in a number of one to several lines, such as, for example, one to three lines, so as to reduce the area actually used for tightening. Moreover, as shown in FIGS. 7 and 8 with respect to the first aspect of the invention, tightening portions may also be formed spirally or in a crisscross fashion.

As mentioned above, if the holder 101 fits too tightly around the wrist, the blood flow may stop to cause the congestion of blood. Therefore, the pressure applied to the skin when the holder 101 is worn on the wrist or ankle with the chemical warmer 102 being accommodated in the accommodating portion 105 is preferably set to 80 hPa or less. If the congestion of blood occurs, heat will accumulate in the afflicted area, possibly resulting in a low-temperature burn; therefore, the pressure is more preferably 40 hPa or less, and still more preferably 35 hPa or less. Also, if the holder 1 becomes loose, it will be difficult to position the holder 1 on the area to be treated because the holder 1 will lose conformity to the afflicted area and easily move; therefore, the lower limit of the pressure is 10 hPa, preferably 20 hPa, and more preferably 30 hPa.

In this embodiment, the holder 101 has a bag shape with a multilayer structure, in which a single sheet of a stretchable textile product (such as a woven, knitted, nonwoven, or like cloth) is folded into two layers, and the open regions are closed. The opening 106 is provided in the knitting process. While the number of openings 106 may be one as in this embodiment, it may be one to six, and preferably one to three, depending on the area to be treated or the position thereof. While the position of the opening 106 is not particularly limited, when the holder 101 is long, or the chemical warmer 102 is small, it may be arranged vertically or horizontally in the longitudinal direction and/or lateral direction of the accommodating portion 105, or the position may be diagonally staggered.

As shown in FIG. 18, the opening 106 is made in the knitting process by leaving open a portion of an outer cloth 110a of the bag-shaped holder 101. In this case, the accommodating portion 105 is a space between the outer cloth 110a and an inner cloth 110b that constitute the holder 101, in which the chemical warmer 102 is accommodated.

Although not illustrated, the bag-shaped holder 101 may be sewn appropriately so that an individual accommodating portion (pocket) is formed. In this case, if the pocket is disposed on an inner or outer bending portion of the joint, the wearer will feel pressure during joint flexure, so that the pocket is preferably formed on an upper side of a limb (on the same side as the back of the hand, or on the same side as the back of the foot for use on the ankle) so as not to prevent the motion of the joint. Depending on the area to be treated, however, a pocket may additionally be disposed so as to surround the wrist, and/or may be disposed in the longitudinal direction of the arm.

Although not illustrated, an accommodating portion may be prepared separately, and then sewn on or attached to the holder 101. When attaching an accommodating portion, an adhesive may be used, or hook-and-loop fasteners or the like may be used so as to allow the position of the accommodating portion to be detachably changed.

In addition, the opening 106 may be provided with a lid or a cover, or may be provided with buttons or fasteners (zippers or hook-and-loop fasteners) in order to prevent the chemical warmer from dropping off.

The holder 101 is composed of a stretchable material. The material used may be composed of a synthetic fiber alone or a mixture of synthetic fibers and natural fibers such as vegetable fibers, animal fibers, mineral fibers, and the like. Such materials may be suitably selected in consideration of stretchability, wearing comfort, heat conductivity, workability, wear resistance, chemical resistance, and the like. Examples of preferable fiber materials include polyamide, polyurethane, polyester and the like. Although not limited, the thickness of the fiber may be from 3 to 100 μm, and preferably from 10 to 50 μm. Mechanical properties of the fiber are preferably such that the tensile strength is from 10 to 300 kg/mm², and preferably from 10 to 100 kg/mm², and the elongation percentage is from 1 to 50%, and preferably from 5 to 30%.

While the stretchability also depends on the properties of the fiber, it can be controlled by selecting the method of cloth production. The cloth is preferably knitted by a method such as circular knitting, weft knitting, plain knitting, rib knitting, purl knitting, interlock knitting or the like, with circular knitting being particularly preferable because a circular-knitted cloth can be preformed into a tubular shape without sewing.

The holder 101 is prepared using the above-described stretchable material. Any method can be used for preparing the holder 1 other than the above-described method of using a single sheet or using a bag-shaped sheet, as long as stretchability and flexibility are not impaired.

Moreover, in order to improve the wearing comfort and softness to the skin, a backlining layer (not illustrated) composed of a cotton (towel) fabric, a linen fabric, or a silk fabric may be formed on the body-side of the inner cloth 110b of the holder 101.

The tightening portion 109 is preferably prepared by using the same fabric as that of the holder, but changing the knitting method to impart a tightening force, or by using a rubber material. The method is not particularly limited, however, as long as it can impart a tightening force. For example, hook-and-loop fasteners or a tightening belt, or a combination of these may alternatively be used instead of the above-mentioned methods.

The holder 101 may be made available in various sizes so that a suitable size can be selected according to the body size of a potential user.

Alternatively, as shown in FIGS. 9 to 11 with respect to the first aspect of the invention, the holder 101 may comprise a mechanism for adjusting the size of the holder according to the body size of the user.

The chemical warmer used as another element of the invention is now described referring to FIG. 19.

For example, as shown in FIG. 19, the chemical warmer 102 used in the invention may comprise a flat bag (hereinafter referred to as an “inner bag”) 111; an exothermic composition 112 that is sealed in the inner bag 111 and that oxidizes and generates heat in the presence of air; and a hermetic bag (hereinafter referred to as an “outer bag”) 113 housing the flat bag 111.

The exothermic composition 112 is preferably dispersed in the inner bag 11 in an amount of 0.1 to 1 g per 10 mm square, so as to achieve even and mild heating.

The outer bag 113 is preferably composed of a porous film such as polyethylene, polypropylene, a silica-evaporated film, a vinylidene chloride coating film or the like. Among these examples, a vinylidene chloride coating film is preferred in view of its impermeability to oxygen and water vapor and an appropriate permeability to hydrogen. The thickness of the porous film is preferably from 30 to 300 μm.

A known exothermic composition may be used as the exothermic composition 112 that is sealed inside the flat bag 111 and that oxidizes and generates heat in the presence of air. Examples of usable exothermic compositions include, but are not limited to, those comprising metallic particles such as iron particles, activated carbon, water, water-holding agents (such as wood flour, vermiculite, diatomite, perlite, silica gel, alumina, water-absorbing resins and the like), salt, and the like.

At least one portion of the inner bag 111 is an air-permeable surface with a group of small air holes or micropores. That is to say, the surface 114 of the inner bag 111 may be air-permeable, and the other surface 115 may be air-impermeable, or both the surfaces may be air-permeable. Moreover, a portion of one surface may be an air-permeable surface, with the remaining portion being an air-impermeable surface.

When an air-impermeable surface is provided, it is preferably composed of, for example, a synthetic resin, such as nylon, polyethylene, polypropylene or the like.

The air-permeable surface is composed of a known material, such as a woven cloth, a nonwoven cloth, a knitted cloth, a perforated film of any of the aforementioned air-impermeable materials, or a combination thereof.

In the invention, the size of the inner bag 111 is not particularly limited, and may be selected to suit the shape, size or the like of the chemical warmer accommodating portion (or pocket). The shape of the inner bag 111 is also not limited to a rectangular shape, but may be circular, oval, flap-shaped, heart-shaped, or the like. The thickness of the inner bag 111 is from 1 to 15 mm, and preferably 2 to 8 mm, so as to provide satisfactory wearing comfort while not likely to hinder the motion of the joint. The thickness of the inner bag 111 may be even, or may be uneven to vary the feeling of pressure.

The air permeability of the inner bag 111 may be determined in consideration of the skin surface temperatures at which so-called low-temperature burns may occur and the time therefor. For example, low-temperature burns may occur in six hours at a skin temperature of 42° C., in three hours at a skin temperature of 43° C., in 1.5 hours at a skin temperature of 44° C., and in 45 minutes at a skin temperature of 45° C. The air permeability may be controlled while considering the composition of the exothermic composition so that these thresholds are not exceeded. In the case of short-period treatments, however, the heating temperature may further be elevated (for example, to 60° C., or even higher to 70° C.).

While the air permeability of the entire inner bag 111 is not limited, the water vapor permeability as measured according to Method A (Humidity Sensor Method) defined in JIS (Japanese Industrial Standard) K 7129 is preferably 100 to 2,000 g/m²·24 h, and more preferably 200 to 800 g/m²·24 h. If the water vapor permeability exceeds 2,000 g/m²·24 m, the exothermic composition may generate excessive heat, whereas if it is lower than 100 g/m²·24 h, the exothermic composition may not be able to generate sufficient heat.

The heating temperature and duration can be controlled by changing the structures of the chemical warmer 102 and the accommodating portion 105, and in particular, the structures of the inner cloth 110b and the outer cloth 110a of the accommodating portion 105, and also by changing the material of the backlining layer.

Examples of structural combinations include, but are not limited to, the following.

(1) Cases Where the Inner Cloth Is Air-Permeable, and the Chemical Warmer Is Air-Permeable on Both Surfaces Thereof In these cases, a relatively high heating temperature is achieved, but care needs to be taken concerning low-temperature burns and the like. When necessary, a backlining layer may be provided to further control the air permeability.

(2) Cases Where the Inner Cloth Is Air-Permeable, and the Chemical Warmer Is Air-Permeable on Only One Surface Thereof In these cases, air intake is facilitated by placing the chemical warmer in the accommodating portion 105 so that the air-permeable surface is positioned on the side of the accommodating portion opposite to the body (outer side), thereby making the heating temperature relatively high (about 38 to about 42° C.). In contrast, air intake is restricted by placing the chemical warmer in the accommodating portion 105 so that the air-permeable surface is positioned on the body side (inner side) of the accommodating portion, thereby making the heating temperature relatively low (about 36 to about 40° C.).

That is to say, the heating temperature can be adjusted to a temperature necessary for thermotherapy by changing the direction in which the chemical warmer 102 is placed.

(3) Cases Where the Inner Cloth Is Air-Impermeable or Substantially Air-Impermeable, and the Chemical Warmer Is Air-Permeable on Both Surfaces Thereof

In these cases, a relatively high heating temperature can be achieved, and the heating temperature can be controlled by selecting the heat conductivity, thereby avoiding low-temperature burns and the like.

(4) Cases Where the Inner Cloth Is Air-Impermeable or Substantially Air-Impermeable, and the Chemical Warmer Is Air-Permeable on Only One Surface

In these cases, as in cases (2) above, the heating temperature can be adjusted to a temperature necessary for thermotherapy by changing the direction in which the chemical warmer 102 is placed, while considering the heat conductivity of the inner cloth.

(5) Cases Where Both the Inner Cloth and the Outer Cloth Are Air-Impermeable, and Means for Placing And Removing the Chemical Warmer Is Provided to the Accommodating Portion In these cases, the function of the accommodating portion enables temperature adjustments necessary for thermotherapy.

Examples of means to make the inner cloth air-impermeable or substantially air-impermeable include attaching an air-impermeable film to the body side of the accommodating portion.

In one embodiment, the chemical warmer may simply be fastened (attached) to the holder, rather than making the accommodating portion of the chemical warmer into a bag shape (or pocket) as described above. In this case, the chemical warmer is attached to the outer or inner surface of the holder. Examples of means for attaching the chemical warmer include attaching with adhesive, fixing with hook-and-loop fasteners, fixing with buttons, and the like.

When a chemical warmer is attached to the holder, an adhesive layer composed of a common adhesive material may be formed on one surface of the inner bag 111 (for example, on the surface 115 in FIG. 19), and a release paper or a release film may be formed on the adhesive layer.

The adhesive layer may be formed entirely on one surface of the inner bag 111, or may be partially formed in suitable patterns such as stripes, checks, dots or the like. The adhesive layer may also be formed either on an air-impermeable surface or an air-permeable surface.

Although a thermotherapy device for use on the wrist has been described above, it can also be used for the ankle by adjusting the shape and the size to the ankle.

In addition, a thermotherapy device for the ankle encompassed in the second aspect of the invention comprises a tubular holder composed of a stretchable material and a chemical warmer held in the holder; the holder comprising an ankle region and a back-and-heel region; and the ankle region having an accommodating portion in which the chemical warmer can be placed and removed therefrom, or to which the chemical warmer can be attached.

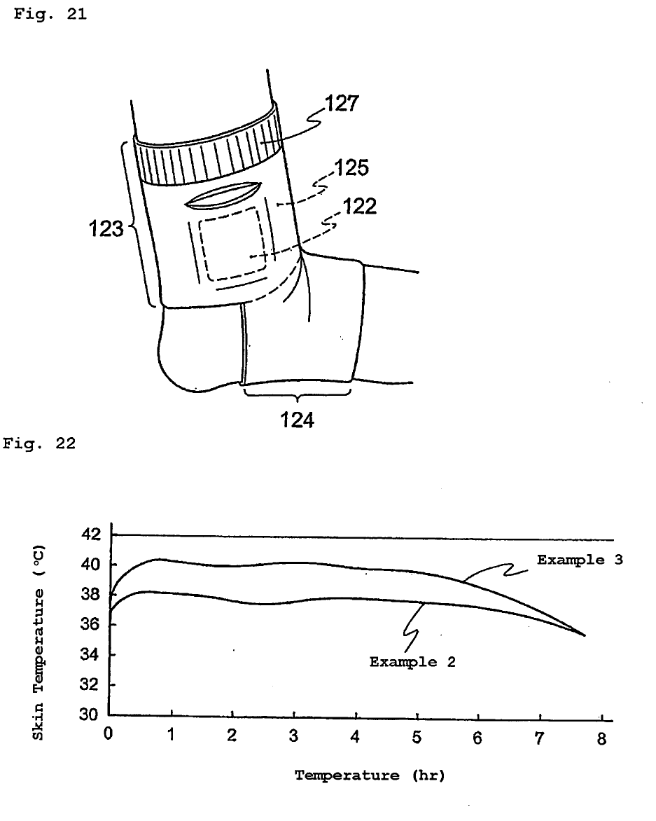

Referring to FIGS. 20 and 21, a preferred embodiment of the thermotherapy device for the ankle according to the invention will be described. The present invention, however, is not limited to this embodiment, and various modifications can be made thereto.

FIG. 20 shows a schematic diagram for use in illustrating the thermotherapy device for the ankle of the invention; and FIG. 21 shows a diagram for use in illustrating the thermotherapy device of FIG. 20 worn on the ankle.

The thermotherapy device for the ankle comprises a tubular holder 121 and a chemical warmer 122. The holder 121 has an ankle region 123 and a back-and-heel region 124.

The ankle region 123 of the holder 121 has a bag-shaped accommodating portion 125 in which the chemical warmer 122 can be placed and removed therefrom. The accommodating portion 125 is provided with an opening 126 through which the chemical warmer can be placed and removed. Although FIG. 20 shows the single opening 126 provided on a side of the ankle, another opening 126 may additionally be provided on the opposite side thereto, or one to six of such openings 126 may be provided as needed, depending on the area to be treated. A tightening portion 127 is also provided.

The holder 121 for the ankle is provided with a partial cutout 128, which is disposed at the heel to enable the holder to be stably worn by the wearer (see FIG. 21).

Instead of providing the cutout 128, the holder 121 may be produced on a circular knitting machine for socks or the like so as to conform to the protrusion of the heel, or may be provided beforehand with a hole in a portion corresponding to the heel.

The materials of and the methods for producing the holder 121, the shapes and the arrangements of the tightening portion, the mechanisms for adjusting the size of the holder, the backlining layer, and the chemical warmer 122 described in the thermotherapy device for the wrist can also be applied to the thermotherapy device for the ankle. Moreover, the thermotherapy device for the ankle according to the second aspect of the invention may be provided as an extension portion of socks.

The thermotherapy device of the present invention will next be described with reference to Examples, which are not intended to limit the invention.

### EXAMPLE 1

A holder for the knee (circumference: 180 mm, length: 150 mm) comprising a bag-shaped chemical warmer accommodating portion with two openings as shown in FIG. 1 was prepared using a 0.5-mm thick Spandex cloth made by circular knitting a polyamide fiber.

The holder was worn by an adult male on the knee like a supporter. At that time, a thermocouple was attached to the skin that presses against each chemical warmer through the inner cloth of the holder, so as to record changes in the skin surface temperature.

Two chemical warmers, each having an inner bag that measured 95 mm in length, 70 mm in width and 3 mm in thickness, and each having one air-impermeable surface, were used. Each inner bag was removed from the outer bag and shaken in the air several times. One of the chemical warmers was then placed in one pocket with its air-impermeable surface facing downward (and its air-permeable surface facing upward), and the other chemical warmer was placed in the other pocket with its air-impermeable surface facing upward (and its air-permeable surface facing downward), and changes in the skin temperature over time (eight hours) were examined. The results are shown in FIG. 14. In FIG. 14, the solid line represents the side on which the chemical warmer was set with its air-impermeable surface facing downward, and the dashed line represents the side on which the chemical warmer was set with its air-permeable surface facing upward.

As shown in FIG. 14, the temperature of the skin can be adjusted by selecting whether the air-impermeable surface faces upward or downward.

The user performed an ordinary daily routine wearing the thermotherapy device of the invention for eight hours. As a result, the thermotherapy device did not greatly deviate in position, and the chemical warmers also did not drop off. Moreover, the user did not feel uncomfortable in extending or bending the knee, and no disorders such as the congestion of blood occurred.

### EXAMPLE 2

A holder for the wrist (joint circumference: 140 mm, length: 130 mm) comprising a bag-shaped chemical warmer accommodating portion with a single opening as shown in FIG. 15 was prepared using a 0.5 mm thick Spandex made by circular knitting a polyamide fiber.

The holder was worn by an adult male on the wrist like a supporter. At that time, a thermocouple was attached to the skin that presses against a chemical warmer through the inner cloth, so as to record changes in the skin surface temperature.

A chemical warmer having an inner bag that measured 95 mm in length, 70 mm in width and 3 mm in thickness, and having one air-impermeable surface, was used. The inner bag was removed from the outer bag and placed in the accommodating portion through the opening, with its air-impermeable surface facing downward (and its air-permeable surface facing upward), and changes in the skin temperature over time (eight hours) were examined. The results are shown in FIG. 22.

### EXAMPLE 3

The same conditions as in Example 2 were used except that a chemical warmer was placed in the accommodating portion with its air-impermeable surface facing upward (and its air-permeable surface facing downward) , and changes in the skin temperature over time (eight hours) were examined. The results are shown in FIG. 22.

As shown in FIG. 22, the temperature of the skin can be adjusted by selecting whether the air-impermeable surface faces upward or downward.

The user performed an ordinary daily routine wearing the thermotherapy device of the invention for eight hours. As a result, the thermotherapy device did not greatly deviate in position, and the chemical warmer also did not drop off. Moreover, the user did not feel uncomfortable in rotating or bending the wrist, and no disorders such as the congestion of blood occurred.

### INDUSTRIAL APPLICABILITY

The thermotherapy device of the present invention can maintain temperatures suitable for thermotherapy for a long period, remains in place despite joint motion, does not cause the congestion of blood due to pressure, and can hold a heat-generating member in a suitable area of the knee, elbow, wrist, or ankle.

1: A thermotherapy device worn on a joint comprising a tubular holder composed of a stretchable material and a chemical warmer held in the holder; the holder having an accommodating portion in which the chemical warmer can be placed and removed therefrom, or to which the chemical warmer can be attached.

2: A thermotherapy device worn on a knee or elbow comprising a tubular holder composed of a stretchable material and a chemical warmer held in the holder; the holder having an accommodating portion in which the chemical warmer can be placed and removed therefrom, or to which the chemical warmer can be attached; and the thermotherapy device being worn on the knee or elbow so that the chemical warmer accommodating portion is positioned around a leg or an arm.

3: The thermotherapy device according to claim 2, wherein the pressure applied to the skin when the holder is worn on the knee or elbow with the chemical warmer being accommodated in the holder is 80 hPa or less.

4: The thermotherapy device according to claim 2, wherein the holder has a bag shape with a multilayer structure, and the chemical warmer accommodating portion is an integral part of the bag.

5: The thermotherapy device according to claim 4, wherein the chemical warmer accommodating portion is formed without any partition provided in the holder.

6: The thermotherapy device according to claim 4, wherein the chemical warmer accommodating portion is a pocket formed with a partition in the bag-shaped holder.

7: The thermotherapy device according to claim 2, wherein the chemical warmer comprises an inner bag in which only one surface is air-permeable.

8: The thermotherapy device according to claim 2, wherein the chemical warmer comprises an inner bag in which both surfaces are air-permeable.

9: A thermotherapy device worn on a wrist comprising a tubular holder composed of a stretchable material and a chemical warmer held in the holder; the holder comprising a wrist region and a palm-and-back-of-the-hand region; the holder having an accommodating portion in which the chemical warmer can be placed and removed therefrom, or to which the chemical warmer can be attached; and the palm-and-back-of-the-hand region having an opening through which a finger is inserted.

10: A thermotherapy device worn on an ankle comprising a tubular holder composed of a stretchable material and a chemical warmer held in the holder; the holder comprising an ankle region and a back-and-heel region; and the holder having an accommodating portion in which the chemical warmer can be placed and removed therefrom, or to which the chemical warmer can be attached.

11: The thermotherapy device according to claim 10, wherein the pressure applied to the skin when the holder is worn on the wrist or ankle with the chemical warmer being accommodated in the holder is 80 hPa or less.

12: The thermotherapy device according to claim 10, wherein the holder has a bag shape with a multilayer structure, and the chemical warmer accommodating portion is an integral part of the bag.

13: The thermotherapy device according to claim 12, wherein the chemical warmer accommodating portion is formed without any partition provided in the holder.

14: The thermotherapy device according to claim 12, wherein the chemical warmer accommodating portion is a pocket formed with a partition in the bag-shaped holder.

15: The thermotherapy device according to claim 10, wherein the chemical warmer comprises an inner bag in which only one surface is air-permeable.

16: The thermotherapy device according to claim 10, wherein the chemical warmer comprises an inner bag in which both surfaces are air-permeable.

17: The thermotherapy device according to claim 9, wherein the pressure applied to the skin when the holder is worn on the wrist or ankle with the chemical warmer being accommodated in the holder is 80 hPa or less.

18: The thermotherapy device according to claim 9, wherein the holder has a bag shape with a multilayer structure, and the chemical warmer accommodating portion is an integral part of the bag.

19: The thermotherapy device according to claim 18, wherein the chemical warmer accommodating portion is formed without any partition provided in the holder.

20: The thermotherapy device according to claim 18, wherein the chemical warmer accommodating portion is a pocket formed with a partition in the bag-shaped holder.
